(19) United States
(12) Plant Patent Application Publication
(10) Pub. No.: US 2003/0079258 P1
(43) Pub. Date: Apr. 24, 2003

(54) Ground cover rose variety 'POULeas'

(76) Inventors: L. Pernille Olesen, Fredensborg (DK); Mogens N. Olesen, Fredensborg (DK)

Correspondence Address: MOGENS N OLESEN, POULSEN ROSER PACIFIC INC, 2550 OLD MILITARY ROAD, CENTRAL POINT, OR, 97502, US

(21) Appl. No.: 10/189,148

(22) Filed: Jul. 2, 2002

Related U.S. Application Data
(63) Continuation of application No. 09/273,168, filed on Mar. 19, 1999, now abandoned.

(51) Int. Cl. A01H 5/00

(52) U.S. Cl. PLT/107

(57) ABSTRACT

A new garden rose plant which has abundant, pink flowers and attractive foliage. This new and distinct variety has shown to be uniform and stable in the resulting generations from asexual propagation.

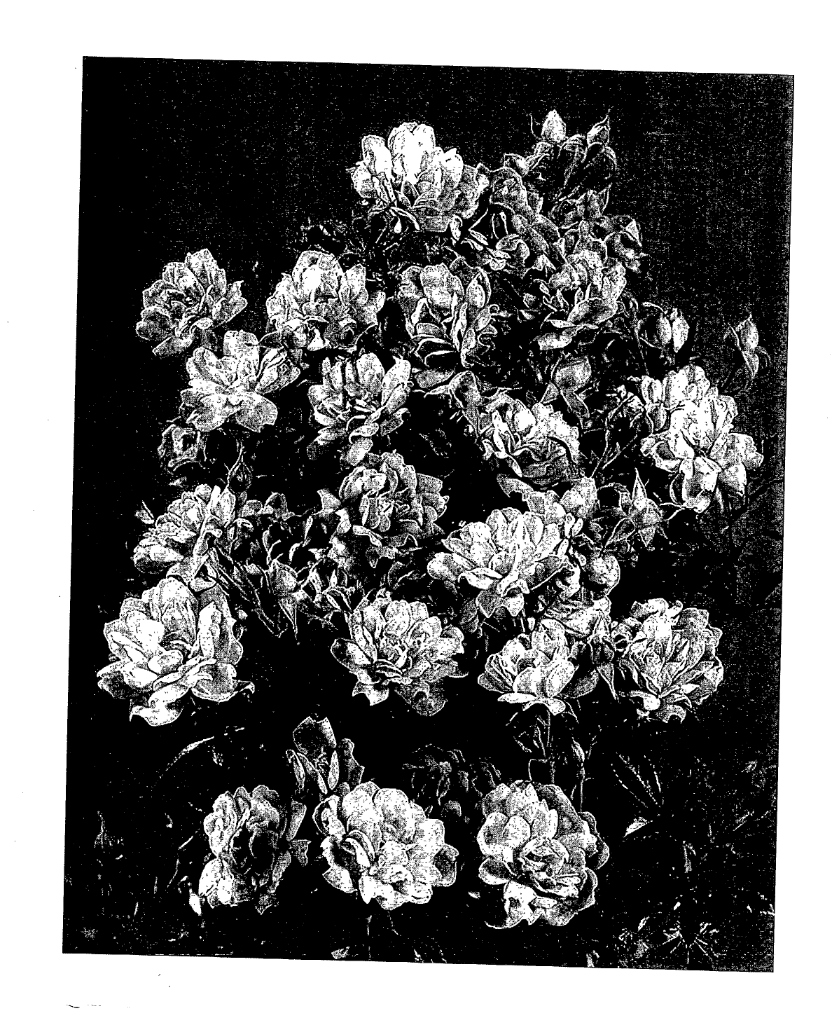

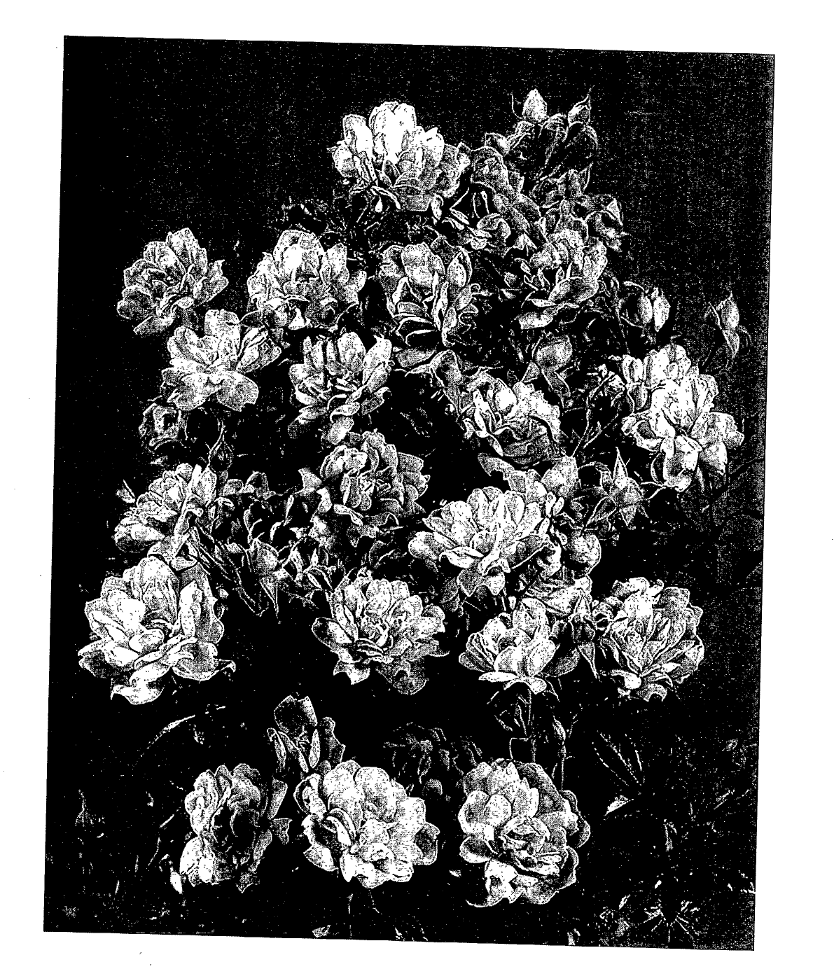

### SUMMARY OF THE INVENTION

The present invention constitutes a new and distinct variety of garden rose plant which of originated from a controlled crossing between an unnamed seedling and ‘POULover’. The two parents were crossed in 1988 and resulting seeds were planted in a controlled environment. The new variety is named ‘POULeas’. The new rose may be distinguished from its seed parent, an unnamed seeding, by the following combination of characteristics:

1. The seed parent has soft pink double flowers which are present in sprays;

2. The habit on the unnamed seedling is more ball like and less spreading than ‘POULeas’.

The new variety may be distinguished from its pollen parent, ‘POULover’, created by the same inventors, by the following combination of characteristics:

1. The pollen parent is a climber with long upright canes, while ‘POULeas’ is an extremely compact shrub rose;

2. The pollen parent provides limited repeat flowering during a season.

The objective of the hybridization of this rose variety for nursery and garden use was to create a new and distinct variety with unique qualities, such as:

1. Abundant, pink flowers which are present on the plant almost continually throughout the growing season;

2. A ground cover rose with uniform growth and a spreadinig habit;

3. Glossy and diseise resistant foliage which requires limited maintenance, making it ideal for use in landscapes

4. Good growth on its own roots as well its a traditionally budded plant.

This combination of qualities is not present in previously available commercial cultivars of this type and distinguish ‘POULeas’ from all other varieties of which we are aware.

As part of their rose development program, L. Pernille Olesen and Mogens N. Olesen germinated the seeds from the aforementioned hybridization and conducted evaluations on the resulting seedlings in a controlled environment in Fredensborg, Denmark.

‘POULeas’ was selected by the inventors in the spring of 1989 as a single plant from the progeny of the aforementioned hybridization.

Asexual reproduction of ‘POULeas’ by cuttings and traditional budding was first done by L. Pernille and Mogens N. Olesen in August 1989, in their nursery in Fredensborg, Denmark. This initial and other subsequent propagations conducted in controlled environments have demonstrated that the characteristics of ‘POULeas’ are true to type and are transmitted from one generation to the next.

### BRIEF DESCRIPTION OF THE DRAWING

The accompanying color illustration shows as true as is reasonably possible to obtain in color photographs of this type, the typical characteristic of the buds, flowers, leaves, stems, and a plant of ‘POULeas’. Specifically illustrated in SHEEET 1:

1. Stem showing branching and the attachment of leaves, buds, and peduncles;

2. Flower bud, partially opened bud, and open bloom;

3. Flower petals, detached;

4. Sepals, receptacle, ancl pedicel;

5. Flowering stem is well as a bare stem exhibiting thorns;

6. Leaves.

### DETAILED DESCRIPTION OF THE VARIETY

The following is a description of ‘POULeas’, as observed in its outdoor growtih in a field nursery in Jackson County, Oregon. Observations were conducted during October, 1998. Color references are made using The Royal Horticultural Society (London, England) Colour Chart, 1995, except where common terms of color are used.

For a comparison, several physical characteristics of the rose variety ‘POULurt’, a yellow, compact ground cover rose variety from the same inventors described and ilustrated in U.S. Plant Pat. No. 9,637 and issued on Sep. 3, 1996 are compared to ‘POULeas’ in Chart 1.

CHART 1

| | ‘POULeas’ | ‘POULurt’ |
|---|---|---|
| Color upon | Red Group 55A. | Yellow-Orange Group |
| opening. Upper | | 15A. |
| Surface of flower | | |
| petal. | | |
| Color upon | Red Group 54B. | Yellow-range Group |
| opening. Reverse | | 15A, becoming Yellow- |
| side of flower | | Orange Group 14A |
| petal. | | towards base. |

Parents:

Seed parent.—Unmaned, unpatented seedling.

Pollen parent.—‘POULover’.

Classification:

Botanical.—Rosa hybrida.

Commercial.—Ground Cover.

### FLOWER AND FLOWER BUD

Bloominig habit: Nearly continuous.

Flower bud:

Size.—Upon opening, 18 mm in length from base of receptacle to end of bud.

Bud form.—Short, pointed ovoid.

Bud color.—As sepals unfold, Red Group 46A. Where the outerside of the petals is exposed to sunlight while sepals are unfolding, color is Red Group 46A. Where the outerside of the petals is not exposed to sunlight while sepals are unfolding, color is Red Group 51A, with intonations of Yellow-Green Group 145C. Red Group 54B at ¼ opening.

Sepals.—Yellow-Green Group 144A. Moderate foliaccous appendages on three of the five sepals. Surfaces of sepals Slightly pubescent on exterior and moderately pubescent on interior of sepal. Moderate numbers of stipitate glaids are present on outerside and margins of the sepals.

Receptacle.—Surface: Smooth. Shape: Urn-shaped. Size: 5 mm (h)×4 mm (w). Color: Yellow-Green Group 144B.

Peduncle.—Surface: Smooth, glabrous. Length: 20 mm to 30 mm average length. Color: Yellow-Green Group 144B. with intonations of Greyed-Purple 185D. Strength: Upright.

Borne.—Multiple buds per stem. Normally with 3-7 buds per flowering stem.

Flower bloom:

Fragrance.—Faint. Fresh and sweet.

Duration.—As a cut flower 2 to 3 days. The blooms have a duration on the plant of approximately 3 to 4 days. Petals fall cleanly away from plant. Individual flowers relatively short lived, but the flowers open sequentially on each flowering stem providing a long duration of bloom.

Size.—Average flower diameter is 30-35 mm when open.

Form.—

Shape of flower when viewed from the side.—Upon opening, upper part: Flattened convex Upon opening, lower part: Flat Open flower, upper part: Convex Open flower, lower part: Concave.

Petalage.—Semi-double. Average range: 15-22 petals under normal conditions with 0-3 petaloids.

Color:

Upon opening, petals.—Outermost petals: Upper Surface: Red Group 55A Reverse Side: Red Group 54B Innermost petals Upper surface: Red Group 55A Reverse Side: Red Group 54A.

Upon opening, basal petal spots.—Outermost petals Outer Side: Green-Yellow Group 1D. Inner Side: Green-Yellow Group 1A. Innermost petals: Outer Side: Green-Yellow Group 1C. Inner Side: Green-Yellow Group 1B.

After openiing, petalls.—Outermost petals: Upper Surface: Red Group 55B. Reverse Side: Red Group 55B. Innermost petals: Upper Surface: Red Group 55B. Reverse Side: Red Group 55B.

After opening, basal petal spots.—Petals: Outer Side: White Group 155A. Inner Side: White Group 155A.

General Tonality: On open flower Red Group 55B. No change in the general tonality at the end of the 3rd day. Afterwards, general tonality is Red Group 55C-55D.

Petals:

Petal reflex.—Somewhat.

Petal edge.—With point in center of margin.

Shape.—Deltoid shaped.

Petaloids.—0-3.

Thickness.—Average.

Arrangement.—Informal.

Reproductive Organs:

Pollen.—Color: Yellow-Orange Group 22A. Abundance: Limited.

Anther.—Size: Small. Color: Green-Yellow Group 1D. Abundance: Average

Filaments.—Color: Yellow-Green Group 154C.

Stigmas.—Slightly inferior in location to anthers. Color: Yellow-Green Group 145D With top of stigmas haviig intonation of Red Group 55C.

Styles.—Color: Yellow-Green Group 145A.

Hips.—None observed.

### PLANT

Plant growth: Compact and bushy. When grown as a budded field grown plant on Rosa multiflora understock, the average height of the plant itself is 45 cm and the average width is 60 cm.

Stems:

Color.—Young wood: Yellow-Green Group 146B. Older wood: Yellow-Green Group 146B.

Thorns.—Incidence: Moderate Size: Average length: 4 mm. Color: Yellow-Green Group 151A. Shape: Concave.

Surface.—Young wood: Smooth. Older wood: Smooth.

Plant foliage: Normal number of leaflets on normal leaves in middle of the stem: 5-7 leaflets.

Leaf size.—Small, 55 mm (l)×30 mm (w).

Abudance.—Very dense foliage.

Color.—Upper Leaf Surface: Green Group 137A. Lower Leaf Surface: Green Group 137C. Juvenile foliage: Upper surface coloration is Green Group 137A. Lower surface coloration is Green Group 137A. Anthocyanin intonation: None.

Plant leaves and leaflets:

Stipules.—Size: 12 mm (l)×5 mm (w). Color: Green Group 137C.

Presence of stipitate glands: Limited number along margins, slightly glaucous underneath.

Petiole.—Length: 18 mm. Color: Green Group 137B. Underneath: Green Group 137C. WIth fine pickles and some stipitate glands. Margins: Limited number of stipitate glands.

Rachis.—Color: Green Group 137A. Underneath: Fine prickles. Margins: Very limited stipitate glands.

Leaflet.—Edge: Serrated. Shape: Lanccolate. Texture: Glossy.

Disease resistance: Exceptional resistance to mildew, black spot, and Botrytis under normal growing conditions in Jackson County, Oregon. In two years in trial none of the above mentioned diseases have been observed in a field which has never been sprayed with fungicide.

Heat and Cold resistance: ‘POULeas’ has been found to be resistant to damage from cold, heat and drought damage in USDA Zone 7.

1. A new and distinct variety of rose plant of the shrub class, substantially as herein illustrated and described as a distinct and novel rose variety due to its abundant, pink flowers, vigorous growth, disease resistance, and extended period of bloom.
